(19) United States
(12) Patent Application Publication
(10) Pub. No.: US 2021/0379135 A1
(43) Pub. Date: Dec. 9, 2021

(54) APPLICATION OF LITCHI CHINENSIS PERICARP EXTRACT MAINLY COMPOSED OF POLYMER POLYPHENOLS IN PREPARATION OF ALPHA AMYLASE INHIBITOR

(71) Applicant: Guangxi Zhuang Autonomous Region and Chinese Academy of Science, Guangxi Institute of Botonay, Guilin (CN)

(72) Inventors: Ziming YANG, Guangxi (CN); Li ZHANG, Guangxi (CN)

(73) Assignee: Guangxi Zhuang Autonomous Region and Chinese Academy of Science, Guangxi Institute of Botonay, Guilin (CN)

(21) Appl. No.: 17/002,760

(22) Filed: Aug. 25, 2020

Related U.S. Application Data
(63) Continuation of application No. PCT/CN2020/100763, filed on Jul. 8, 2020.

(30) Foreign Application Priority Data
Jun. 8, 2020 (CN) 202010513290.5

(51) Int. Cl. A61K 36/77; A01N 65/08

(52) U.S. Cl. CPC A61K 36/77; A01N 65/08; A61K 2236/53; A61K 2236/51; A61K 2236/333

(57) ABSTRACT

The present invention discloses an application of litchi chinensis pericarp extract mainly composed of polymer polyphenols in preparation of α-amylase inhibitor, which belongs to the technical field of biology. The litchi chinensis pericarp extract mainly composed of polymer polyphenols disclosed by the present invention has a very good inhibitory effect on α-amylase, whereas α-amylase is applied in weight and blood glucose reduction, biological pesticides, dental caries prevention, and osteoporosis prevention and treatment.

### TECHNICAL FIELD

The present invention relates to the technical field of biology, more particularly to an application of litchi chinensis pericarp extract mainly composed of polymer polyphenols in preparation of α-amylase inhibitor.

### BACKGROUND

α-amylase inhibitor can be widely used: (1) as drugs, health products or food for prevention and treatment of obesity and diabetes: starch is a main component of carbohydrate in diet, which is hydrolyzed by saliva and pancreatic amylase to produce maltose and then absorbed by the intestinal tract in the form of glucose. α-amylase inhibitor can inhibit the activity of amylase in the gastrointestinal tract, and slow down the digestion and absorption of the main carbohydrate starch in food, thereby inhibiting the increase in blood glucose concentration, which can effectively cooperate with the dietary treatment of diabetic patients. For obese patients, α-amylase inhibitor can reduce the conversion of sugar to fat, delay the emptying of the intestinal tract, and increase the consumption of fat to reduce the weight. α-amylase inhibitor plays a role in weight and blood glucose reduction through the inhibitory effect on amylase and is excreted through the gastrointestinal tract, so it does not need to enter the blood circulation system, does not act on the brain center, and has no side effects when used in high dosages, which is in line with the weight reducing principles of the World Health Organization; (2) as a new insect-resistant and insecticidal biological pesticide: the α-amylase in an insect body decomposes starch, glycogen and other substances into maltose, glucose and fructose that can be directly absorbed to participate in the energy metabolism in the insect body. α-amylase inhibitor inhibits the activity of α-amylase, which will reduce the assimilation of carbohydrate in the insect body, thereby inhibiting the growth and development of an insect, and even causing the death of the insect, so it can be used as a new insect-resistant and insecticidal biological pesticide; (3) for preventing dental caries: α-amylase inhibitor can also prevent the saccharification of starchy foods in the mouth and reduce the tartar formed by carbohydrate, thereby preventing dental caries; and (4) for preventing and treating osteoporosis: α-amylase inhibitor can increase the residual carbohydrate in the intestinal tract, which is favorable for the survival of beneficial bacteria in the intestinal tract, and ferment the residual carbohydrate to produce lactic acid, which makes the intestinal tract tend to be acidic, thereby promoting the dissolution and absorption of calcium in food, and producing the effect of preventing and treating osteoporosis.

Litchi chinensis is the fruit of an evergreen plant of Sapindaceae, which is mainly produced in Guangdong, Guangxi, Fujian and other places in China. At present, the annual output of litchi chinensis in China is about 1.6 million tons, accounting for more than 80% of the world's total. During the deep processing of litchi chinensis, pericarp waste accounting for about 20% of the mass of fresh fruit is generated. As a bulk agricultural product waste, litchi pericarp has not been effectively developed and utilized so far.

Therefore, the problem to be urgently solved by those skilled in the art is to provide an application of litchi chinensis pericarp extract mainly composed of polymer polyphenols in preparation of α-amylase inhibitor.

### SUMMARY

In view of this, the present invention provides an application of litchi chinensis pericarp extract mainly composed of polymer polyphenols in preparation of α-amylase inhibitor.

To achieve the above purpose, the present invention adopts the following technical solution:

An application of litchi chinensis pericarp extract mainly composed of polymer polyphenols in preparation of α-amylase inhibitor.

Further, a method for preparing the litchi chinensis pericarp extract mainly composed of polymer polyphenols is as follows:

(1) extracting fresh litchi chinensis pericarp and 80% ethanol water solution with a volume of 8 times of that of the fresh litchi chinensis pericarp twice at room temperature, soaking for 7 days each time, filtering, and merging the filtrate;

(2) centrifuging the filtrate at 3,500 rpm for 10 minutes to collect a supernatant and obtain a drug solution;

(3) purifying the drug solution by column chromatography through XDA-7 macroporous resin and eluting the drug solution by an ethanol water solution with a volume fraction of 60% to obtain an extract eluent; the column chromatography purification conditions are that: the mass concentration of loading buffer is 3.5 mg/mL, the pH value of the drug solution is 4.5, the sample loading rate is 4 BV/h, and the flow rate of the eluent is 6 BV/h; and

(4) concentrating the extract eluent under reduced pressure at a vacuum degree of 95 kPa, a heating bath temperature of 40° C. and a cooling water temperature of 15° C., and spray-drying the resulting reflux liquid at a feeding rate of 51 mL/min, an inlet air temperature of 160° C., an exhaust air temperature of 60° C., a feed liquid concentration of 22%, and an atomizer rotating speed of 21,000 r/min to obtain the litchi chinensis pericarp extract.

Further, the litchi chinensis pericarp extract mainly composed of polymer polyphenols is applied in preparation of drugs for reducing weight, reducing blood glucose, preventing dental caries, and preventing and treating osteoporosis.

Further, the litchi chinensis pericarp extract mainly composed of polymer polyphenols is applied in preparation of biological pesticides.

The specific application method of the litchi chinensis pericarp extract mainly composed of polymer polyphenols is that: the litchi chinensis pericarp extract mainly composed of polymer polyphenols, with or without the addition of auxiliary materials, is prepared into various preparations according to conventional methods; specifically, the extract can be made into existing conventional dosage forms such as capsules, tablets, granules and water solution.

It can be known from the above technical solution that compared with the prior art, the present invention discloses and provides an application of litchi chinensis pericarp extract mainly composed of polymer polyphenols in preparation of α-amylase inhibitor. Litchi chinensis pericarp contains a large amount of polyphenols which have a plurality of biological activities such as antioxidant activity, anti-tumor activity, cardiovascular disease preventing activity and anti-aging activity. The present invention proves, by study, that the litchi chinensis pericarp extract mainly composed of polymer polyphenols has a very good inhibitory effect on α-amylase, whereas α-amylase is applied in weight and blood glucose reduction, biological pesticides, dental caries prevention, and osteoporosis prevention and treatment.

### DESCRIPTION OF DRAWINGS

To more clearly describe the technical solution in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a content proportion chart of each polar component of the litchi chinensis pericarp extract of the present invention; and

FIG. 2 is an inhibition curve of the litchi chinensis pericarp extract mainly composed of polymer polyphenols of the present invention with different concentrations on amylase.

### DETAILED DESCRIPTION

The technical solution in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

### Embodiment 1

A method for preparing the litchi chinensis pericarp extract, comprising the following specific steps:

Extracting fresh litchi chinensis pericarp and 80% ethanol water solution with a volume of 8 times of that of the fresh litchi chinensis pericarp twice at room temperature, soaking for 7 days each time, filtering, and merging the filtrate; centrifuging the filtrate at 3,500 rpm for 10 minutes to collect a supernatant and obtain a drug solution; purifying the drug solution by column chromatography through XDA-7 macroporous resin and eluting the drug solution by an ethanol water solution with a volume fraction of 60% to obtain an extract eluent; the column chromatography purification conditions are that: the mass concentration of loading buffer is 3.5 mg/mL, the pH value of the drug solution is 4.5, the sample loading rate is 4 BV/h, and the flow rate of the eluent is 6 BV/h; and concentrating the extract eluent under reduced pressure at a vacuum degree of 95 kPa, a heating bath temperature of 40° C. and a cooling water temperature of 15° C., and spray-drying the resulting reflux liquid at a feeding rate of 51 mL/min, an inlet air temperature of 160° C., an exhaust air temperature of 60° C., a feed liquid concentration of 22%, and an atomizer rotating speed of 21,000 r/min to obtain the litchi chinensis pericarp extract.

The content of polyphenols is determined by the Folin-phenol colorimetric method, and is 77.8%.

### Embodiment 2

A component separation study is carried out to the litchi chinensis pericarp extract obtained from embodiment 1, and the specific operations are as follows:

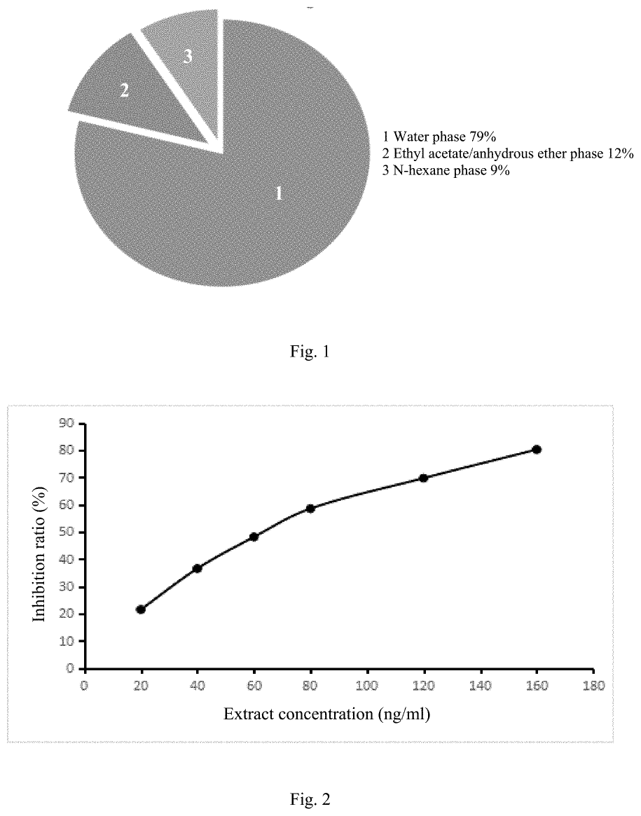

Extracting the litchi chinensis pericarp extract obtained from embodiment 1 stepwise with n-hexane and ethyl acetate/anhydrous ether (1:1) to obtain an n-hexane phase, an ethyl acetate/anhydrous ether phase and a water phase of the litchi chinensis pericarp extract; and determining the total content of phenols in each of the three phases respectively. The results show that the total content of phenols in the water phase is the highest, followed by that in the ethyl acetate/anhydrous ether phase, and the total content of phenols in the n-hexane phase is the lowest (FIG. 1). The phenols contained in the water phase are polyphenol polymers, while those in the n-hexane phase and the ethyl acetate/anhydrous ether phase are polyphenol oligomers and monomers. Therefore, the litchi chinensis pericarp extract is mainly composed of polyphenol polymers, followed by oligomers and monomers, wherein polymer polyphenols account for about 79%.

### Embodiment 3: Inhibitory Effect of the Litchi Chinensis Pericarp Extract Mainly Composed of Polymer Polyphenols on α-Amylase

(I) Preparation of Test Reagents

(1) Preparation of 0.01 Mol/L PBS Solution with pH=7.0:

Adding 61.0 ml of 0.2 mol/L disodium hydrogen phosphate, 39.0 ml of 0.2 mol/L sodium dihydrogen phosphate, and 0.8% sodium chloride solution to obtain 1 L of mixture, thus a 0.01 mol/L PBS buffer is obtained.

0.2 mol/L disodium hydrogen phosphate: taking 35.81 g of Na₂HPO₄.12H₂O, adding 500 ml of distilled water, and preserving after fully dissolved;

0.2 mol/L sodium dihydrogen phosphate: taking 15.6 g of NaH₂PO₄.2H₂O, adding 500 ml of distilled water, and preserving after fully dissolved; and

Preparation of 0.8% sodium chloride solution: taking 4 g of NaCl, adding 500 ml of distilled water, and preserving after fully dissolved.

(2) Preparation of 0.5% starch solution: taking 200 mg of soluble starch, dissolving the starch in 40 ml of 0.01 mol/L PBS buffer with pH=7.0, heating to make the starch dissolved, and preserving after fully dissolved.

(3) Preparation of α-amylase solution: adding α-amylase to 0.01 mol/L PBS buffer with pH=7.0 to prepare a 10 μg/ml solution, and preserving after fully dissolved.

(4) Preparation of DNS developer: taking 10.5 g of sodium hydroxide, adding an appropriate amount of water to dissolve the sodium hydroxide, taking 91.0 g of potassium sodium tartrate, adding an appropriate amount of water to dissolve the potassium sodium tartrate, heating properly to help dissolving if necessary, and blending the above two solutions uniformly after fully dissolved; weighing 3.15 g of DNS (3,5-dinitrosalicylic acid), adding the DNS to the above solution, and heating to make the DNS fully dissolved; adding 2.5 ml of phenol after cooled down, adding 2.5 g of anhydrous sodium sulfite, stirring fully to blend uniformly, and adding distilled water to prepare 500 ml of solution which can be used after standing for 3 days.

(5) Preparation of solutions of the litchi chinensis pericarp extract mainly composed of polymer polyphenols: dissolving the litchi chinensis pericarp extract mainly composed of polymer polyphenols in 0.01 mol/L PBS buffer with pH=7.0 to prepare solutions of the litchi chinensis pericarp extract mainly composed of polymer polyphenols with various concentrations.

(II) Determination of Inhibitory Activity of the Litchi Chinensis Pericarp Extract Mainly Composed of Polymer Polyphenols on α-Amylase

Blank group: adding 0.9 ml of 0.01 mol/L PBS buffer with pH=7.0, adding 0.1 ml of α-amylase solution, blending uniformly, and incubating in a 37° C. thermotank with water bath for 10 minutes; adding 0.2 ml of starch solution to start reaction, incubating in a 37° C. thermotank for 10 minutes, adding 1 ml of DNS developer, blending uniformly, and incubating in a 95° C. thermotank for 5 minutes; cooling with running water, and determining OD value with a semi-automatic biochemical analyzer at a wavelength of 540 nm.

Determination group: adding 0.8 ml of 0.01 mol/L PBS buffer with pH=7.0, adding 0.1 ml of solutions of the litchi chinensis pericarp extract mainly composed of polymer polyphenols with different concentrations and 0.1 ml of α-amylase solution, blending uniformly, and incubating in a 37° C. thermotank with water bath for 10 minutes; adding 0.2 ml of starch solution to start reaction, incubating in a 37° C. thermotank for 10 minutes, adding 1 ml of DNS developer, blending uniformly, and incubating in a 95° C. thermotank for 5 minutes; cooling with running water, and determining OD value with a semi-automatic biochemical analyzer at a wavelength of 540 nm.

Control group: adding 0.8 ml of 0.01 mol/L PBS buffer with pH=7.0, adding 0.1 ml of solutions of the litchi chinensis pericarp extract mainly composed of polymer polyphenols with different concentrations and 0.1 ml of 0.01 mol/L PBS buffer with pH=7.0, blending uniformly, and incubating in a 37° C. thermotank with water bath for 10 minutes; adding 0.2 ml of starch solution to start reaction, incubating in a 37° C. thermotank for 10 minutes, adding 1 ml of DNS developer, blending uniformly, and incubating in a 95° C. thermotank for 5 minutes; cooling with running water, and determining OD value with a semi-automatic biochemical analyzer at a wavelength of 540 nm.

Calculating the inhibition ratio of the litchi chinensis pericarp extract mainly composed of polymer polyphenols with different concentrations on α-amylase, and the result is shown in FIG. 2. The result in FIG. 2 shows that when the concentration of the extract is increased unceasingly, the inhibitory effect on α-amylase is increased; when the concentration of the litchi chinensis pericarp extract mainly composed of polymer polyphenols is 151.5 μg/ml, the inhibition ratio on α-amylase is 80.2%, so it can be seen that the litchi chinensis pericarp extract mainly composed of polymer polyphenols has a very good inhibitory effect on α-amylase. Calculated by SPSS17.0 calculation software, the half maximal inhibitory concentration (IC50) of the litchi chinensis pericarp extract mainly composed of polymer polyphenols on α-amylase is 62.2 μg/ml.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. An application of litchi chinensis pericarp extract mainly composed of polymer polyphenols in preparation of α-amylase inhibitor.

2. The application of litchi chinensis pericarp extract mainly composed of polymer polyphenols in preparation of α-amylase inhibitor according to claim 1, wherein a method for preparing the litchi chinensis pericarp extract mainly composed of polymer polyphenols is as follows:
(1) extracting fresh litchi chinensis pericarp and 80% ethanol water solution with a volume of 8 times of that of the fresh litchi chinensis pericarp twice at room temperature, soaking for 7 days each time, filtering, and merging the filtrate;
(2) centrifuging the filtrate at 3,500 rpm for 10 minutes to collect a supernatant and obtain a drug solution;
(3) purifying the drug solution by column chromatography through XDA-7 macroporous resin and eluting the drug solution by an ethanol water solution with a volume fraction of 60% to obtain an extract eluent; the column chromatography purification conditions are that: the mass concentration of loading buffer is 3.5 mg/mL, the pH value of the drug solution is 4.5, the sample loading rate is 4 BV/h, and the flow rate of the eluent is 6 BV/h; and
(4) concentrating the extract eluent under reduced pressure at a vacuum degree of 95 kPa, a heating bath temperature of 40° C. and a cooling water temperature of 15° C., and spray-drying the resulting reflux liquid at a feeding rate of 51 mL/min, an inlet air temperature of 160° C., an exhaust air temperature of 60° C., a feed liquid concentration of 22%, and an atomizer rotating speed of 21,000 r/min to obtain the litchi chinensis pericarp extract.

3. The application of litchi chinensis pericarp extract mainly composed of polymer polyphenols in preparation of α-amylase inhibitor according to claim 1, wherein the litchi chinensis pericarp extract mainly composed of polymer polyphenols is applied in preparation of drugs for reducing weight, reducing blood glucose, preventing dental caries, and preventing and treating osteoporosis.

4. The application of litchi chinensis pericarp extract mainly composed of polymer polyphenols in preparation of α-amylase inhibitor according to claim 1, wherein the litchi chinensis pericarp extract mainly composed of polymer polyphenols is applied in preparation of biological pesticides.
